(19) United States
(12) Patent Application Publication
(10) Pub. No.: US 2010/0240624 A1
(43) Pub. Date: Sep. 23, 2010

(54) Ophthalmic Formulations of Ketotifen and Methods of Use

(75) Inventors: Matthew J. Chapin, Amesbury, MA (US); Mark B. Abelson, Andover, MA (US); George Minno, Suwanee, GA (US); Jackie Nice, Medford, MA (US); Paul Gomes, Haverhill, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Aciex Therapeutics, Inc., Westborough, MA (US)

(21) Appl. No.: 12/701,130

(22) Filed: Feb. 5, 2010

Related U.S. Application Data
(60) Provisional application No. 61/160,918, filed on Mar. 17, 2009.
(60) Provisional application No. 61/174,675, filed on May 1, 2009.

(51) Int. Cl. A61K 31/4535; A61K 31/568; A61P 27/02; A61P 27/04; A61P 27/14

(52) U.S. Cl. 514/171; 514/324

(57) ABSTRACT

The present invention provides topical formulations of ketotifen that provide a comfortable formulation when instilled in the eye and are effective in the treatment and prevention of ocular allergy, particularly allergic conjunctivitis. The invention further provides methods of treating and preventing ocular allergy by in a subject in need of such treatment by topical application of the ketotifen formulations of the invention to the eye of a subject in need thereof.

### REFERENCE TO RELATED APPLICATIONS

This nonprovisional application claims priority to U.S. Provisional Application Ser. No. 61/160,918, filed Mar. 17, 2009, and U.S. Provisional Application Ser. No. 61/174,675, filed May 1, 2009, the contents of which are each hereby incorporated by reference in their entireties.

### FIELD OF THE INVENTION

The invention relates to ophthalmic formulations comprising ketotifen and methods for treating and preventing ocular allergy.

### BACKGROUND OF THE INVENTION

There exists a need for topical ophthalmic pharmaceutical products to effectively treat ocular allergy, particularly allergic conjunctivitis, a disorder that is characterized by the clinical signs and symptoms of eye itching, redness, chemosis, tearing, and swelling, and is estimated to have a prevalence of over 20% in the United States. The signs and symptoms of allergic conjunctivitis can significantly impact the quality of life of patients, from social interactions, productivity at work and school, to the ability to perform visual tasks such as working on a computer or reading.

The mast cell is the primary cell involved in eye allergy, and when stimulated by an allergen (pollen, dust, dander) releases a host of substances that produce the signs and symptoms of allergic conjunctivitis (itching, redness, swelling, and tearing). Histamine is the primary mediator released and stimulates receptors on nerve endings and blood vessels to produce itching and redness. There are two histamine receptors that have been identified on the ocular surface. H1 receptors on nerve endings lead to itching, and H1 and H2 receptors on blood vessels lead to dilation of the blood vessels, leading to redness, and leakage of fluid from the vessels into the surrounding tissue producing swelling. Allergic conjunctivitis may also co-exist with other external ocular conditions and diseases, such as dry eye, or irritations caused by pollutants or other causes. This leads to a compromised tear film, which serves to protect the ocular surface from allergens.

Currently available treatments for ocular allergy include: drops which can wash allergens off the ocular surface and act as a barrier for the eye (e.g. artificial tears), drugs which block histamine from binding to the histamine receptors (e.g. antihistamines), drugs that block the release of histamine and other substances from the mast cell (e.g. mast cell stabilizers), drugs with multiple modes of action (e.g. antihistamine/mast cell stabilizing agents), steroids, NSAIDs, and drugs that can actively constrict blood vessels thus reducing redness and swelling (e.g. vasoconstrictors). The criteria which may be considered in evaluating the appropriateness of an agent for a patient include: efficacy at onset of action, duration of action, how well it controls the individual signs and symptoms of allergic conjunctivitis, and comfort of the drop when instilled in the eye. The comfort of an ophthalmic product depends on the active pharmaceutical ingredient itself, as well as the nature of the formulation and the vehicle that makes up the product. For example, oral antihistamines have been shown to induce decreased tear production and lead to dryness of the ocular surface, making the eye susceptible to irritation by an ophthalmic product.

Ketotifen fumarate is a pharmaceutical agent having antihistamine, mast-cell stabilizing, and anti-inflammatory properties. It is currently approved in the United States as 0.025% ketotifen fumarate ophthalmic solution under the trade names Alaway™ (Bausch and Lomb) and Zaditor™ (Novartis), as well as under other names as it was approved for sale over-the-counter. Ketotifen is also approved in other countries, including Japan and South America, as a 0.05% formulation.

In the United States, the 0.025% formulation is indicated for the temporary prevention of itching of the eye due to allergic conjunctivitis and for twice daily dosing. In controlled clinical studies it has been shown to have a duration of action up to 12 hours. It is generally known that the higher concentration formulations of ketotifen fumarate that are available outside the United States (i.e. 0.05%) are not as comfortable when instilled in the eye and produce stinging/burning on the ocular surface. Various ophthalmic formulations of ketotifen have been described. For example, U.S. Pat. No. 6,774,137 describes a ketotifen formulation comprising 0.01-0.04% ketotifen fumarate and having an osmolarity in the range of 210 to 290 mOsm. U.S. Patent Application Publication No. 20060148899 describes an ophthalmic ketotifen formulation comprising 0.01-0.05% ketotifen fumarate and having an osmolarity in the range of 400 to 875 mOsm. U.S. Patent Application Publication No. 20050239745 describes an ophthalmic ketotifen formulation comprising 0.025-0.10% ketotifen fumarate in which the ketotifen is formulated with a tear substitute for increased comfort at the higher concentrations. U.S. Patent Application Publication No. 20070048389 describes an ophthalmic ketotifen formulation comprising 0.01-0.10% ketotifen fumarate containing hydrogen peroxide as a preservative. More comfortable formulations of ketotifen fumarate at concentrations above 0.025% are desirable because they would offer increased efficacy and/or would require less frequent administration (e.g., once a day) to achieve the desired therapeutic effects.

Naphazoline (in the hydrochloride form) is the common name for 2-(1-naphthylmethyl)-2-imidazoline hydrochloride. It is a sympathomimetic agent with marked alpha adrenergic activity. It is used as a vasoconstrictor in various ophthalmic formulations such as Visine A™ (0.025%) and Albalon™, Nafazir™, Napheon™, AND Opcon™, each of which contains naphazoline at a concentration of 0.10%. U.S. Patent Application Publication No. 20070208058 describes stabilized ophthalmic formulations of ketoifen (0.001-0.20%) and naphazoline (0.001-0.20%) having a pH of less than 5.

Fluticasone is a potent synthetic corticosteroid. Existing products are formulated for nasal administration for the treatment of asthma and allergic rhinitis, or as creams and ointments for the treatment of eczema and psoriasis. U.S. Patent Application Publication No. 20060263350 generally describes a formulation comprising an antihistamine, which may be ketotifen, and a steroid, which may be fluticasone, for the treatment of allergic rhinitis.

Oxymetazoline is a selective alpha-1 agonist and partial alpha-2 agonist topical decongestant, used in the form of oxymetazoline hydrochloride in commercially available nasal sprays. Oxymetazoline has sympathomimetic properties, and thus constricts the blood vessels of the nose and sinuses via activation of alpha-2 adrenergic receptors.

### SUMMARY OF THE INVENTION

The present invention provides comfortable topical ophthalmic formulations for the treatment and prevention of ocular allergy, particularly allergic conjunctivitis. Also provided for are products that contain a combination of ingredients which act synergistically to relieve the signs and symptoms of ocular allergy, particularly ocular itching, redness, swelling, and nasal symptoms. In particular, the formulations described herein provide ketotifen, or a pharmaceutically acceptable salt thereof, suitable for ophthalmic use in a comfortable ophthalmic formulation when instilled in the eye. The invention also provides methods for the treatment and prevention of ocular allergy, particularly allergic conjunctivitis, in a subject in need of such treatment, by topically administering a ketotifen formulation of the invention directly to the eye of the subject.

The present invention is based, in part, on the surprising discovery that a marginal increase in concentration of ketotifen over that of currently marketed ketotifen ophthalmic solutions, produces more than a marginal increase in efficacy, as defined by hours of itching relief. In a particular embodiment, the invention provides ophthalmic formulations of ketotifen as the only active agent in the formulation for treating and preventing ocular allergy, particularly allergic conjunctivitis. Such formulations as described herein provide superior efficacy and comfort profiles over ketotifen formulations previously approved for ocular allergy (e.g., Zaditor™, a 0.025% ketotifen solution, and a 0.05% ketotifen solution marketed outside the U.S.), with less frequent dosing required than the formulations currently marketed for ocular allergy. Remarkably, one drop daily of the ketotifen formulations described herein is capable of relieving the signs and symptoms of ocular allergy (e.g., ocular itching) for at least 16 hours, and up to 24 hours. More remarkably, the ketotifen formulations described and tested herein compare more favorably to currently sold ketotifen products with as much as twice the API concentration but duration to only support BID dosing (i.e., twice daily), whereas the efficacy of the ketotifen formulations of the present invention supports QD dosing (i.e., once a day).

The invention also provides ophthalmic formulations of ketotifen in combination with one or more additional active ingredients selected from oxymetazoline, naphazoline and fluticasone. Such combination formulations are effective in further mitigating the symptoms of ocular allergy, especially allergic conjunctivitis, such as redness, chemosis, lid swelling and nasal symptoms.

In some embodiments, the ophthalmic formulations of the invention comprise a tear substitute, or component thereof. In a particular embodiment, the tear substitute, or component thereof, comprises hydroxypropylmethyl cellulose (Hypromellose or HPMC). According to some embodiments, the concentration of HPMC ranges from about 0.5% to about 2% w/v, or any specific value within said range. According to some embodiments, the concentration of HPMC ranges from about 0.5% to about 1% w/v, or any specific value within said range. In a preferred embodiments, the concentration of HPMC ranges from about 0.7% to about 0.9% w/v, or any specific value within said range (i.e., about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

In another particular embodiment, the tear substitute, or component thereof, comprises carboxymethyl cellulose (CMC). According to some embodiments, the concentration of CMC ranges from about 0.5% to about 2% w/v, or any specific value within said range. According to some embodiments, the concentration of CMC ranges from about 0.5% to about 1% w/v, or any specific value within said range. In a preferred embodiments, the concentration of CMC ranges from about 0.7% to about 0.9% w/v, or any specific value within said range (i.e., about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

In yet another particular embodiment, the ophthalmic formulations of the invention comprise a polymeric, mucoadhesive vehicle. Examples of mucoadhesive vehicles suitable for use in the methods or formulations of the invention include but are not limited to aqueous polymeric suspensions comprising one or more polymeric suspending agents including without limitation dextrans, polyethylene glycol, polyvinylpyrolidone, polysaccharide gels, Gelrite®, cellulosic polymers, and carboxy-containing polymer systems. In a particular embodiment, the polymeric suspending agent comprises a crosslinked carboxy-containing polymer (e.g., polycarbophil). Examples of cross-linked carboxy-containing polymer systems suitable for use in the topical ophthalmic formulations of the invention include but are not limited to Noveon AA-1, Carbopol®, and/or DuraSite® (InSite Vision).

According to some embodiments, the ophthalmic formulations of the present invention has a viscosity that ranges from about 30 to about 150 centipoise (cpi), preferably about 50 to about 120 cpi, even more preferably about 60 to about 115 cpi (or any specific value within said ranges). According to preferred embodiments, the ophthalmic formulations of the present invention has a viscosity that ranges from about 60 to about 80 cpi, or any specific value within said range (i.e., about 60 cpi, about 61 cpi, about 62 cpi, about 63 cpi, about 64 cpi, about 65 cpi, about 66 cpi, about 67 cpi, about 68 cpi, about 69 cpi, about 70 cpi, about 71 cpi, about 72 cpi, about 73 cpi, about 74 cpi, about 75 cpi, about 76 cpi, about 77 cpi, about 78 cpi, about 79 cpi, or about 80 cpi).

In a particular embodiment, the ketotifen formulations of the invention do not comprise a tear substitute or component thereof, a polymeric, mucoadhesive vehicle, or any other type of masking agent (i.e., an agent used to cover-up, disguise or alleviate any stinging or burning sensation in the eye caused upon topical administration of ketotifen to the eye).

Preferably, the ketotifen formulations of the invention are comprised of ketotifen fumarate.

In a particular embodiment, the ketotifen formulations of the invention further comprises glycerol. In a specific embodiment, the concentration of glycerol in the formulation is from 2% to 3% (v/v), or any specific value within said range.

Optionally, the ketotifen formulations of the invention further comprise a preservative, preferably benzalkonium chloride or stabilised oxychloro complex) (Purite°. In a specific embodiment, the concentration of benzalkonium chloride in the formulation is from 0.005% to 0.02% (v/v), or any specific value within said range (e.g., 0.01%). In one embodiment, the pH of the formulation is between 5.0 and 7, preferably between 5.0 and 6.5, and most preferably about 5.5. In one embodiment, the formulation is an aqueous formulation. In one embodiment, the formulation is in the form of a single dose unit which does not contain a preservative.

In one embodiment, the invention provides an ophthalmic formulation comprising ketotifen (or a pharmaceutically acceptable salt thereof) as the only active agent in the formulation, wherein the concentration of ketotifen is from 0.01% to 0.20%, preferably from 0.02% to 0.04% (w/v) (or any specific value within said range), the pH of the formulation is between 5.5 and 7, the formulation does not comprise a tear substitute or component thereof, a polymeric, mucoadhesive vehicle, or hydrogen peroxide, and wherein the osmolality of the formulation is greater than 290 mOsm and less than 400 mOsm. In a particular aspect of this embodiment, the ketotifen is in the form of ketotifen fumarate. Optionally, the formulation further comprises a preservative, preferably benzalkonium chloride or stabilised oxychloro complex (Purite®).

In a preferred embodiment, the ketotifen formulation of the invention comprises 0.481 mg/mL ketotifen fumarate (equivalent to 0.350 mg/mL ketotifen base) as the only active agent in the formulation, 0.10 mg/mL benzalkonium chloride, 21.25 mg/mL glycerol, pH adjusted to 5.5, and purified water q.s. to 1 mL, wherein the formulation does not comprise a tear substitute or component thereof, a polymeric, mucoadhesive vehicle, or hydrogen peroxide, and wherein the osmolality of the formulation is 255 mOsm/kg. In another preferred embodiment, the ketotifen formulation of the invention comprises 0.481 mg/mL ketotifen fumarate (equivalent to 0.350 mg/mL ketotifen base) as the only active agent in the formulation for treating and preventing ocular allergy, particularly allergic conjunctivitis, 0.10 mg/mL benzalkonium chloride, 28.75 mg/mL glycerol, pH adjusted to 5.5, and purified water q.s. to 1 mL, wherein the formulation does not comprise a tear substitute or component thereof, a polymeric, mucoadhesive vehicle, or hydrogen peroxide, and wherein the osmolality of the formulation is 345 mOsm/kg.

In another embodiment, the invention provides an ophthalmic formulation comprising ketotifen, or pharmaceutically acceptable salts thereof, wherein the pH of the formulation is greater than 5 and the osmolality is less than 400 mOsm. In one embodiment, the concentration of ketotifen is from 0.01% to 0.20% (w/v) (or any specific value within said ranges). In one embodiment, the pH of the formulation is between 5.5 and 7. In one embodiment, the osmolality of the formulation is 225 to 390 mOsm. In one embodiment, the formulation further comprises a tear substitute or component thereof. In a specific embodiment, the tear substitute, or component thereof, contains hydroxypropylmethyl cellulose or carboxymethyl cellulose. In a particular embodiment, the hydroxypropylmethyl cellulose or carboxymethyl cellulose is present at a concentration of 0.5% to 1%, preferably 0.7% to 0.9%, and the resulting viscosity of the formulation is approximately 60-80 cpi. In one embodiment, the formulation further comprises a preservative, preferably benzalkonium chloride or stabilised oxychloro complex)(Purite°. In one particular embodiment, the formulation comprises carboxymethyl cellulose and stabilised oxychloro complex)(Purite°.

In another embodiment, the invention provides an ophthalmic formulation comprising ketotifen and naphazoline, or pharmaceutically acceptable salts thereof, wherein the pH of the formulation is greater than 5 and the osmolality is less than 400 mOsm. In one embodiment, the ketotifen is in the form of ketotifen fumarate. In one embodiment, the naphazoline is in the form of naphazoline hydrochloride. In one embodiment, the concentration of ketotifen is from 0.01% to 0.20% (w/v) (or any specific value within said ranges). In one embodiment, the concentration of naphazoline is from 0.001% to 0.20% (w/v) (or any specific value within said range). In one embodiment, the pH of the formulation is between 5.5 and 7. In one embodiment, the osmolality of the formulation is 225 to 390 mOsm. In one embodiment, the formulation further comprises a tear substitute, or component thereof. In a specific embodiment, the tear substitute, or component thereof, contains hydroxypropylmethyl cellulose or carboxymethyl cellulose. In a particular embodiment, the hydroxypropylmethyl cellulose or carboxymethyl cellulose is present at a concentration of 0.5% to 1%, preferably 0.7% to 0.9%, and the resulting viscosity of the formulation is approximately 60-80 cpi. In one embodiment, the formulation further comprises a preservative, preferably benzalkonium chloride or stabilised oxychloro complex (Purite®). In one particular embodiment, the formulation comprises carboxymethyl cellulose and stabilised oxychloro complex (Purite®).

In another embodiment, the invention provides an ophthalmic formulation comprising ketotifen and fluticasone, or pharmaceutically acceptable salts thereof. In one embodiment, the ketotifen is in the form of ketotifen fumarate. In one embodiment, the concentration of ketotifen is from 0.001% to 0.20% (w/v) (or any specific value within said range). In one embodiment, the concentration of fluticasone is from 0.001% to 0.20% (w/v) (or any specific value within said range). Preferably, the concentration of fluticasone is from 0.001% to 0.01% (w/v) (or any specific value within said range). In a particular embodiment, the concentration of fluticasone is 0.005% (w/v). In one embodiment, the pH of the formulation is between 4 and 7. In one embodiment, the osmolality of the formulation is 225 to 400 mOsm. In another embodiment, the osmolality of the formulation is 400 to 875 mOsm. In one embodiment, the formulation further comprises a tear substitute, or component thereof. In a specific embodiment, the tear substitute, or component thereof contains hydroxypropylmethyl cellulose or carboxymethyl cellulose. In a particular embodiment, the hydroxypropylmethyl cellulose or carboxymethyl cellulose is present at a concentration of 0.5% to 1%, preferably 0.7% to 0.9%, and the resulting viscosity of the formulation is approximately 60-80 cpi. In one embodiment, the formulation further comprises a preservative, preferably benzalkonium chloride or stabilised oxychloro complex (Purite®). In one particular embodiment, the formulation comprises carboxymethyl cellulose and stabilised oxychloro complex (Purite®).

In another embodiment, the invention provides an ophthalmic formulation comprising ketotifen and oxymetazoline, or pharmaceutically acceptable salts thereof. In one embodiment, the ketotifen is in the form of ketotifen fumarate. In one embodiment, the concentration of ketotifen is from 0.001% to 0.20% (w/v) (or any specific value within said range). In one embodiment, the concentration of oxymetazoline is from 0.001% to 0.20% (w/v), or any specific value within said range. In one embodiment, the pH of the formulation is between 4 and 7. In one embodiment, the osmolality of the formulation is 225 to 400 mOsm. In another embodiment, the osmolality of the formulation is 400 to 875 mOsm. In one embodiment, the formulation further comprises a tear substitute, or component thereof. In a specific embodiment, the tear substitute, or component thereof, contains hydroxypropylmethyl cellulose or carboxymethyl cellulose. In a particular embodiment, the hydroxypropylmethyl cellulose or carboxymethyl cellulose is present at a concentration of 0.5% to 1%, preferably 0.7% to 0.9%, and the resulting viscosity of the formulation is approximately 60-80 cpi. In one embodiment, the formulation further comprises a preservative, preferably benzalkonium chloride or stabilised oxychloro complex (Purite®). In one particular embodiment, the formulation comprises carboxymethyl cellulose and stabilised oxychloro complex (Purite®).

The invention also provides methods of treating and preventing the symptoms of ocular allergy by administering a ketotifen formulation of the invention to the eye of a subject in need of such treatment and prevention. Preferably the subject is a human subject. In certain embodiments, the methods of the invention are also effective to treat nasal symptoms associated with ocular allergy.

In one embodiment, the methods of the invention comprise topically administering to the eye of a subject an ophthalmic formulation comprising an effective amount of ketotifen (or a pharmaceutically acceptable salt thereof) as the only active agent in the formulation for treating and preventing ocular allergy, particularly allergic conjunctivitis, wherein the concentration of ketotifen is from 0.01% to 0.20% (w/v) (or any specific value within said range), the pH of the formulation is greater than 5, and the formulation does not comprise a tear substitute or component thereof, a polymeric mucoadhesive vehicle, or hydrogen peroxide.

In a particular embodiment, the method for treating and preventing ocular allergy, particularly allergic conjunctivitis, in a subject in need thereof, comprises topically administering to the eye of a subject an ophthalmic formulation comprising ketotifen (or a pharmaceutically acceptable salt thereof) as the only active agent in the formulation for treating and preventing ocular allergy, particularly allergic conjunctivitis, glycerol, and benzalkonium chloride, wherein the concentration of ketotifen is from 0.01% to 0.20%, preferably from 0.02% to 0.04% (w/v) (or any specific value within said range), the concentration of glycerol in the formulation is from 2% to 3% (v/v), or any specific value within said range, the pH of the formulation is between 5.0 and 7, preferably between 5.0 and 6.5, and most preferably about 5.5, the osmolality of the formulation is greater than 290 mOsm and less than 400 mOsm, and the formulation does not comprise a tear substitute, a polymeric, mucoadhesive vehicle, or hydrogen peroxide.

In another particular embodiment, the method for treating and preventing ocular allergy, particularly allergic conjunctivitis, comprises administering to the eye of a subject in need thereof an ophthalmic formulation comprising an effective amount of ketotifen (or a pharmaceutically acceptable salt thereof) as the only active agent in the formulation for treating and preventing ocular allergy, particularly allergic conjunctivitis, wherein the concentration of ketotifen is about 0.035% (w/v), the pH of the formulation is about 5.5, the osmolality of the formulation is between 300-350 mOsm (or any specific value within said range), and the formulation does not comprise a tear substitute, a polymeric, mucoadhesive vehicle, or hydrogen peroxide.

In a preferred embodiment, the method for treating and preventing ocular allergy, particularly allergic conjunctivitis, in a subject in need thereof comprises topically administering to the eye of a subject a ketotifen formulation comprising 0.481 mg/mL ketotifen fumarate (equivalent to 0.350 mg/mL ketotifen base) as the only active agent in the formulation for treating and preventing ocular allergy, particularly allergic conjunctivitis, 0.10 mg/mL benzalkonium chloride, 21.25 mg/mL glycerol, pH adjusted to 5.5, and purified water q.s. to 1 mL, wherein the formulation does not comprise a tear substitute or component thereof, a polymeric, mucoadhesive vehicle, or hydrogen peroxide, and wherein the osmolality of the formulation is 255 mOsm/kg. In another preferred embodiment, the method for treating and preventing ocular allergy, particularly allergic conjunctivitis, in a subject in need thereof, comprises topically administering to the eye of a subject a ketotifen formulation comprising 0.481 mg/mL ketotifen fumarate (equivalent to 0.350 mg/mL ketotifen base) as the only active agent in the formulation for treating and preventing ocular allergy, particularly allergic conjunctivitis, 0.10 mg/mL benzalkonium chloride, 28.75 mg/mL glycerol, pH adjusted to 5.5, and purified water q.s. to 1 mL, wherein the formulation does not comprise a tear substitute or component thereof, a polymeric, mucoadhesive vehicle, or hydrogen peroxide, and wherein the osmolality of the formulation is 345 mOsm/kg.

In another embodiment, the method comprises administering to the eye of the subject an ophthalmic formulation comprising an effective amount of ketotifen and naphazoline, or pharmaceutically acceptable salts thereof, wherein the pH of the formulation is greater than 5 and the osmolality is less than 400 mOsm. In another embodiment, the method comprises administering to the eye of the subject an ophthalmic formulation comprising an effective amount of ketotifen and fluticasone. In another embodiment, the method comprises administering to the eye of the subject an ophthalmic formulation comprising an effective amount of ketotifen and oxymetazoline.

In one particular embodiment, the ketotifen formulations of the invention are administered twice a day for the treatment and prevention of ocular allergy, particularly allergic conjunctivitis. In another particular embodiment, the ketotifen formulations of the invention are administered once a day for the treatment and prevention of ocular allergy, particularly allergic conjunctivitis.

The invention also provides kits comprising a pharmaceutical composition of ketotifen formulated for ophthalmic use and instructions for such use. Other features and advantages of the invention will become apparent from the following detailed description and claims.

### BRIEF DESCRIPTION OF THE DRAWINGS

FIG. 1 is a line graph depicting the efficacy of a 0.035% ketotifen formulation (255 mOsm/kg) in reducing ocular itching, as compared to a vehicle control.

FIG. 2 is a line graph depicting the drop comfort of a 0.035% ketotifen (255 mOsm/kg) formulation when instilled into the eye of a subject, as compared to a vehicle control

FIG. 3 is a line graph depicting the efficacy of a 0.035% ketotifen formulation (345 mOsm/kg) in reducing ocular itching, as compared to a vehicle control.

FIG. 4 is a line graph depicting the drop comfort of a 0.035% ketotifen formulation (345 mOsm/kg) when instilled into the eye of a subject, as compared to a vehicle control.

### DETAILED DESCRIPTION OF THE INVENTION

The invention is based in part on the discovery that stable topical ophthalmic formulations of ketotifen, without the coincidental use of a tear substitute or component thereof (e.g., a cellulose derivative), are both comfortable when administered to the eye and effective at reducing the symptoms of ocular allergy. Surprisingly, once a day dosing of the ketotifen formulations of the invention is effective to mitigate the symptoms of ocular allergy, particularly ocular itching, for at least 16 hours, and up to 24 hours. Such comfort upon ocular administration and duration of efficacy have never been previously achieved. The extraordinary efficacy of the ketotifen formulations described herein is attributed to the identification of an optimal ketotifen concentration and an optimal osmolality range for the ophthalmic solution, which in combination provide superior comfort and efficacy over previous ketotifen solutions approved for ocular use. The comfortable ophthalmic formulations described herein will increase patient compliance in the use of such formulations for the treatment and prevention of signs and symptoms associated with ocular allergy and associated ocular discomfort.

The invention also features novel pharmaceutical compositions comprising an effective amount of ketotifen, and optionally one or more tear substitutes, or components thereof, in a pharmaceutically acceptable carrier. The ketotifen component provides relief of the symptoms of ocular allergy, and the one or more tear substitute, or component thereof, provides ocular surface protection via enhancement of the tear film (as evident by increased tear film break up time). An effective amount of the formulations may be used to treat and prevent signs and symptoms associated with ocular allergy and/or general eye irritation, and can also be used to treat another eye disorder if it contains a drug for that disorder. Such formulations provide a comfortable ophthalmic formulation when instilled in the eye and have enhanced efficacy and duration of action over formulations of ketotifen that are not combined with such other agents.

The superior efficacy of the combination ketotifen/tear substitute formulations is attributed to, among other things, the synergistic effect of the combination of ingredients in them. The combination of ketotifen and tear substitute, or component thereof, act synergistically to provide a longer dwell time of the ketotifen on the ocular surface, thus increasing duration and efficacy of action, and to prolong the integrity of the tear film thereby providing protection of the ocular surface (e.g., by increasing the tear film break up time and/or the Ocular Protection Index). As such, the combination ketotifen/tear substitute formulations of the invention are comfortable upon instillation into the eye, and may be used for relief of acute or chronic ocular allergy, and are particularly suitable for both intermittent and long term use.

### Formulations

In the context of the present invention, all concentrations for ketotifen are indicated for ketotifen free base. The skilled artisan would recognize that the corresponding concentration for ketotifen fumarate salt can be calculated by multiplying the ketotifen free base concentration by 1.375. For example, without limitation, 0.035% ketotifen free base is equivalent to 0.0481% ketotifen fumarate salt (0.035%×1.375=0.0481%).

In certain embodiments of the invention, ketotifen, or a pharmaceutically acceptable salt thereof, is formulated at a concentration of 0.01% to 0.20% (w/v), or any specific value within said range. In certain embodiments, ketotifen is formulated at a concentration of 0.01% to 0.025%, 0.025% to 0.050%, 0.050% to 0.075%, 0.075% to 0.1%, 0.1% to 0.15%, 0.15% to 0.175%, or 0.175% to 0.2% (or any specific value within said ranges). In particular embodiments, ketotifen is formulated at 0.02%, 0.025%, 0.03%, 0.035%, 0.04%, 0.045% 0.05%, 0.055%, 0.06%, 0.065%, 0.07%, 0.075%, 0.08%, 0.085%, 0.09%. 0.095%, 0.10%, 0.12%, 0.15%, 0.18% or 0.20%. Preferably, ketotifen is in the form of ketotifen fumarate.

In one embodiment, the ketotifen formulation comprises ketotifen as the only active ingredient in the ophthalmic formulation at a concentration of from 0.01% to 0.20% (w/v) (or any specific value within said range) with an osmolality greater than 290 mOsm. In one embodiment, the osmolality is less than 400 mOsm. In another embodiment, the osmolality is greater than 290 mOsm and less than 400 mOsm (or any specific value within said range). In a particular embodiment, the concentration of ketotifen is 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, or 0.10%. In another embodiment, the concentration of ketotifen fumarate is 0.15%, 0.25%, 0.35%, 0.45%, 0.55%, 0.65%, 0.75%, 0.85%, 0.95%, or 0.20%. Preferably, ketotifen is in the form of ketotifen fumarate.

Preferably, the pharmaceutical compositions according to the present invention are formulated as solutions, suspensions, ointments, gels, emulsions, and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semisolid compositions. In one embodiment, the ketotifen formulations of the invention are aqueous formulations. The aqueous formulations of the invention are typically more than 50%, preferably more than 75%, and most preferably more than 90% by weight water. In another embodiment, the ketotifen formulations are lyophilized formulations.

### Active Agents

Ketotifen is the primary active agent in the formulations of the present invention. However, ketotifen may be formulated with other active agents as described herein. For example, ketotifen may be formulated with one or more additional antiallergic agents. The term “antiallergenic agent” refers to a molecule or composition that treats ocular allergy or reduces a symptom of ocular allergy. The term “ocular allergy” refers to any allergic disease of the eye, e.g., seasonal/perennial allergic conjunctivitis, vernal keratoconjunctivitis, giant papillary conjunctivitis, perennial allergic conjunctivitis, urban allergy, and atopic keratoconjunctivitis. The signs and symptoms of ocular allergies include chemosis, eye itching, tearing, redness and swelling. Non-limiting examples of antiallergenic agents include “antihistamines” or drugs which block histamine from binding to the histamine receptors, “mast cell stabilizers” or drugs that block the release of histamine and other substances from the mast cell, “drugs with multiple modes of action” or drugs that are antiallergenic agents having multiple modes of action (e.g. drugs that are antihistamines and mast cell stabilizers, drugs with antihistamine, mast cell stabilizing and anti-inflammatory activity, etc.), steroids, anti-inflammatories, and nonsteroidal anti-inflammatory drugs or “NSAIDs.”

In certain embodiments, ketotifen is formulated with one or more additional active agents selected from a mast cell stabilizer such as nedocromil, iodoxamide, cromolyn, or cromolyn sodium; a non-steroidal anti-inflammatory drug (“NSAID”) such as diclofenac or ketorolac tromethamine; a vasoconstrictor such as naphazoline, antolazine, or tetrahydrozoline; a topical steroid such as fluticasone, beclomethasone, budesonide, diflorasone, triaminicinolone, clobetasol, difluprednate, prednisolone, dexamethasone, halobetasol, or mometasone; an antihistimine such as astemizole, azelastine, bepotastine, bilastine, brompheniramine, chlorpheniramine, clemastine, desloratidine, dexbrompheniramine, diphenhydramine, doxylamine, ebastine, emedastine, epinastine, fexofenadine, hydroxyzine, levocabastine, levocetirizine, loratidine, mequitazine, mizolastine, olopatadine, oxatomide, phenindamine, pheniramine, pyrilamine, terfenidine, and triprolidine; or an alpha-adrenergic agonist such as epinephrine, fenoxazoline, indanazoline, naphazoline, oxedrine, phenylephrine, tefazoline, tetryzoline, tramazoline, tymazoline, oxymetazoline, or xylometazoline.

In certain embodiments, ketotifen is formulated with one or more additional active agents selected from oxymetazoline, naphazoline, and fluticasone.

### Tear Substitutes

The term “tear substitute” refers to molecules or compositions which lubricate, “wet,” approximate the consistency of endogenous tears, aid in natural tear build-up, or otherwise provide temporary relief of dry eye symptoms and conditions upon ocular administration. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxymethyl cellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Systane®, Teargen II®, Tears Naturale®, Liquigel®, Tears Natural II®, Tears Naturale Free®, and TheraTears®; and polyvinyl alcohols such as Akwa Tears®, HypoTears®, Moisture Eyes®, Murine Lubricating®, and Visine Tears®, Soothe®. Tear substitutes may also be comprised of paraffins, such as the commercially available Lacri-Lube@ ointments. Other commercially available ointments that are used as tear substitutes include Lubrifresh PM®, Moisture Eyes PM® and Refresh PM®.

In one preferred embodiment of the invention, the tear substitute comprises hydroxypropylmethyl cellulose (Hypromellose or HPMC). According to some embodiments, the concentration of HPMC ranges from about 0.5% to about 2% w/v, or any specific value within said range. According to some embodiments, the concentration of HPMC ranges from about 0.5% to about 1.5% w/v, or any specific value within said range. According to some embodiments, the concentration of HPMC ranges from about 0.5% to about 1% w/v, or any specific value within said range. According to some embodiments, the concentration of HPMC ranges from about 0.6% to about 1% w/v, or any specific value within said range. In a preferred embodiments, the concentration of HPMC ranges from about 0.7% to about 0.9% w/v, or any specific value within said range (i.e., about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

For example, without limitation, a tear substitute which comprises hydroxypropyl methyl cellulose is GenTeal® lubricating eye drops. GenTeal® (CibaVision—Novartis) is a sterile lubricant eye drop containing hydroxypropylmethyl cellulose 3 mg/g and preserved with sodium perborate. Other examples of an HPMC-based tear are provided.

In another preferred embodiment, the tear substitute comprises carboxymethyl cellulose sodium. For example, without limitation, the tear substitute which comprises carboxymethyl cellulose sodium is Refresh® Tears. Refresh® Tears is a lubricating formulation similar to normal tears, containing a, mild non-sensitizing preservative, stabilised oxychloro complex (Purite™), that ultimately changes into components of natural tears when used.

In a preferred embodiment, the tear substitute, or component thereof, is an aqueous solution having a viscosity in a range which optimizes efficacy of supporting the tear film while minimizing blurring, lid caking, etc. Preferably, the viscosity of the tear substitute, or component thereof, ranges from 30-150 centipoise (cpi), preferably 30-130 cpi, more preferably 50-120 cpi, even more preferably 60-115 cpi (or any specific value within said ranges). In a particular embodiment, the viscosity of the tear substitute, or component thereof, is about 70-90 cpi, or any specific value within said range (for example without limitation, 85 cpi).

Viscosity of the ophthalmic formulations of the invention may be measured according to standard methods known in the art, such as use of a viscometer or rheometer. One of ordinary skill in the art will recognize that factors such as temperature and shear rate may effect viscosity measurement. In a particular embodiment, viscosity of the ophthalmic formulations of the invention is measured at 20° C.+/−1° C. using a Brookfield Cone and Plate Viscometer Model VDV-III Ultra+ with a CP40 or equivalent Spindle with a shear rate of approximately apprx. 22.50+/− apprx 10 (1/sec), or a Brookfield Viscometer Model LVDV-E with a SC4-18 or equivalent Spindle with a shear rate of approximately 26+/− apprx 10 (1/sec)).

In some embodiments, the tear substitute, or component thereof, is buffered to a pH 5.0 to 9.0, preferably pH 5.5 to 8.5, more preferably pH 5 to 6 (or any specific value within said ranges), with a suitable salt (e.g., phosphate salts). In some embodiments, the tear substitute further comprises one or more ingredients, including without limitation, glycerol, propyleneglycerol, glycine, sodium borate, magnesium chloride, and zinc chloride.

### Other Excipients

In some embodiments, the ketotifen formulations of the invention comprise one or more pharmaceutically acceptable excipients. The term excipient as used herein broadly refers to a biologically inactive substance used in combination with the active agents of the formulation. An excipient can be used, for example, as a solubilizing agent, a stabilizing agent, a surfactant, a demulcent, a viscosity agent, a diluent, an inert carrier, a preservative, a binder, a disintegrant, a coating agent, a flavoring agent, or a coloring agent. Preferably, at least one excipient is chosen to provide one or more beneficial physical properties to the formulation, such as increased stability and/or solubility of the active agent(s). A “pharmaceutically acceptable” excipient is one that has been approved by a state or federal regulatory agency for use in animals, and preferably for use in humans, or is listed in the U.S. Pharmacopia, the European Pharmacopia or another generally recognized pharmacopia for use in animals, and preferably for use in humans.

Further examples of excipients include certain inert proteins such as albumins; hydrophilic polymers such as polyvinylpyrrolidone; amino acids such as aspartic acid (which may alternatively be referred to as aspartate), glutamic acid (which may alternatively be referred to as glutamate), lysine, arginine, glycine, and histidine; fatty acids and phospholipids such as alkyl sulfonates and caprylate; surfactants such as sodium dodecyl sulphate and polysorbate; nonionic surfactants such as such as TWEEN®, PLURONICS®, or a polyethylene glycol (PEG) designated 200, 300, 400, or 600; a Carbowax designated 1000, 1500, 4000, 6000, and 10000; carbohydrates such as glucose, sucrose, mannose, maltose, trehalose, and dextrins, including cyclodextrins; polyols such as mannitol and sorbitol; chelating agents such as EDTA; and salt-forming counter-ions such as sodium.

Examples of carriers that may be used in the formulations of the present invention include water, mixtures of water and water-miscible solvents, such as C₁- to C₇₋alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

In a particular embodiment, the carrier a polymeric, mucoadhesive vehicle. Examples of mucoadhesive vehicles suitable for use in the methods or formulations of the invention include but are not limited to aqueous polymeric suspensions comprising one or more polymeric suspending agents including without limitation dextrans, polyethylene glycol, polyvinylpyrolidone, polysaccharide gels, Gelrite®, cellulosic polymers, and carboxy-containing polymer systems. In a particular embodiment, the polymeric suspending agent comprises a crosslinked carboxy-containing polymer (e.g., polycarbophil). Examples of cross-linked carboxy-containing polymer systems suitable for use in the topical ophthalmic formulations of the invention include but are not limited to Noveon AA-1, Carbopol®, and/or DuraSite® (InSite Vision).

In particular embodiments, the ketotifen formulations of the invention comprise one or more excipients selected from among the following: a pharmaceutically acceptable salt or buffering agent, a preservative, a tonicity enhancer, a solubilizer, a viscosity enhancing agent, a demulcent, an emulsifier, a wetting agent, a sequestering agent, and a filler. The amount and type of excipient added to the formulation is in accordance with the particular requirements of the formulation and is generally in the range of from about 0.0001% to 90% by weight.

### Salts, Buffers, and Preservatives

The formulations of the present invention may also contain pharmaceutically acceptable salts, buffering agents, and/or preservatives. Examples of such salts include those prepared from the following acids: hydrochloric, hydrobromic, sulfuric, nitric, phosphoric, maleic, acetic, salicylic, citric, boric, formic, malonic, succinic, and the like. Such salts can also be prepared as alkaline metal or alkaline earth salts, such as sodium, potassium or calcium salts. Examples of buffering agents include phosphate, citrate, acetate, and 2-(N-morpholino)ethanesulfonic acid (MES).

For the adjustment of the pH, preferably to a physiological pH, buffers may especially be useful. The pH of the formulations of the present invention should be maintained within the range of 4.0 to 8.0, more preferably about 4.0 to 6.0, more preferably about 5.0 to 6.0. In a particular embodiment, the pH of the formulations described herein is 5.5. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N—(C₈-C₁₈ alkyl)-N,N-dimethylammonium chloride. Further examples of preservatives include antioxidants such as vitamin A, vitamin E, vitamin C, retinyl palmitate, and selenium; the amino acids cysteine and methionine; citric acid and sodium citrate; and synthetic preservatives such as thimerosal, and alkyl parabens, including for example, methyl paraben and propyl paraben. Other preservatives include octadecyldimethylbenzyl ammonium chloride, hexamethonium chloride, benzethonium chloride, phenol, catechol, resorcinol, cyclohexanol, 3-pentanol, m-cresol, phenylmercuric nitrate, phenylmercuric acetate or phenylmercuric borate, sodium perborate, sodium chlorite, alcohols, such as chlorobutanol, butyl or benzyl alcohol or phenyl ethanol, guanidine derivatives, such as chlorohexidine or polyhexamethylene biguanide, sodium perborate, Polyquad®, Germal®II, sorbic acid, and stabilised oxychloro complex)(Purite°. Preferred preservatives include quaternary ammonium compounds, in particular benzalkonium chloride or its derivative such as Polyquad (see U.S. Pat. No. 4,407,791), alkyl-mercury salts, parabens and stabilised oxychloro complex)(Purite°. Where appropriate, a sufficient amount of preservative is added to the ophthalmic composition to ensure protection against secondary contaminations during use caused by bacteria and fungi.

In particular embodiments, the ketotifen formulations of the invention comprise a preservative selected from among the following: benzalkonium chloride, 0.001% to 0.05%; benzethonium chloride, up to 0.02%; sorbic acid, 0.01% to 0.5%; polyhexamethylene biguanide, 0.1 ppm to 300 ppm; polyquaternium-1 (Omamer M)—0.1 ppm to 200 ppm; hypochlorite, perchlorite or chlorite compounds, 500 ppm or less, preferably between 10 and 200 ppm); stabilized hydrogen peroxide solutions, a hydrogen peroxide source resulting in a weight % hydrogen peroxide of 0.0001 to 0.1% along with a suitable stabilizer; alkyl esters of p-hydroxybenzoic acid and mixtures thereof, preferably methyl paraben and propyl paraben, at 0.01% to 0.5%; chlorhexidine, 0.005% to 0.01%; chlorobutanol, up to 0.5%; and stabilised oxychloro complex (Purite®) 0.001% to 0.5%.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable. Formulations lacking a preservative are also preferred for single dose unit compositions.

### Viscosity Enhancing Agents and Demulcents

In certain embodiments, viscosity enhancing agents may be added to the ketotifen formulations of the invention. Examples of such agents include polysaccharides, such as hyaluronic acid and its salts, chondroitin sulfate and its salts, dextrans, various polymers of the cellulose family, vinyl polymers, and acrylic acid polymers.

In certain embodiments, the ketotifen formulations of the invention comprise ophthalmic demulcents and/or viscosity enhancing polymers selected from one or more of the following: cellulose derivatives such as carboxymethy cellulose (0.02 to 5%) hydroxyethylcellulose (0.02% to 5%), hydroxypropylmethyl cellulose or hypromellose (0.02% to 5%), and methylcelluose (0.02% to 5%); dextran 40/70 (0.01% to 1%); gelatin (0.01% to 0.1%); polyols such as glycerol (0.01% to 5%), polyethylene glycol 300 (0.02% to 5%), polyethylene glycol 400 (0.02% to 5%), polysorbate 80 (0.02% to 3%), propylene glycol (0.02% to 3%), polyvinyl alcohol (0.02% to 5%), and povidone (0.02% to 3%); hyaluronic acid (0.01% to 2%); and chondroitin sulfate (0.01% to 2%).

Viscosity of the ophthalmic formulations of the invention may be measured according to standard methods known in the art, such as use of a viscometer or rheometer. One of ordinary skill in the art will recognize that factors such as temperature and shear rate may effect viscosity measurement. In a particular embodiment, viscosity of the ophthalmic formulations of the invention is measured at 20° C.+/−1° C. using a Brookfield Cone and Plate Viscometer Model VDV-III Ultra+ with a CP40 or equivalent Spindle with a shear rate of approximately apprx. 22.50+/− apprx 10 (1/sec), or a Brookfield Viscometer Model LVDV-E with a SC4-18 or equivalent Spindle with a shear rate of approximately 26+/− apprx 10 (1/sec)).

### Tonicity Enhancers

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm. However, in certain embodiments, the formulations of the invention may have a higher osmolality, in the range of 400 to 875 mOsm.

### Solubilizing Agents

The topical formulation may additionally require the presence of a solubilizer, in particular if one or more of the ingredients tends to form a suspension or an emulsion. Suitable solubilizers include, for example, tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. In a preferred embodiment, the solubilizer is a reaction product of castor oil and ethylene oxide, for example the commercial products Cremophor EL® or Cremophor RH400. Reaction products of castor oil and ethylene oxide have proved to be particularly good solubilizers that are tolerated extremely well by the eye. In another embodiment, the solubilizer is tyloxapol or a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

### Exemplary Formulations of the Invention

In one embodiment, the ketotifen formulation comprises ketotifen, or a pharmaceutically acceptable salt thereof, as the only active ingredient at a concentration of from 0.01% to 0.20% (w/v) (or any specific value within said range), glycerol at 0.5% to 5% (v/v, or any specific value within said range), and water, with a pH greater than 5, and an osmolality greater than 290 mOsm. In a particular embodiment, the concentration of ketotifen is 0.015%, 0.02%, 0.025%, 0.03% 0.035%, 0.04%, 0.045%, 0.05%, 0.055%, 0.06%, 0.065%, 0.07%, 0.075%, 0.08%, 0.085%, 0.09%, 0.095%, or 0.10%. In another embodiment, the concentration of ketotifen is 0.15%, 0.25%, 0.35%, 0.45%, 0.55%, 0.65%, 0.75%, 0.85%, 0.95%, or 0.20%. In a preferred embodiment, the ketotifen is ketotifen fumarate. In one embodiment, the osmolality is less than 400 mOsm. In another embodiment, the osmolality is greater than 290 mOsm and less than 400 mOsm, or any specific value within said range.

In one embodiment, the ketotifen formulation comprises ketotifen, or a pharmaceutically acceptable salt thereof, as the only active ingredient in the formulation at a concentration of from 0.01% to 0.20% (w/v) (or any specific value within said range), glycerol at 0.5% to 5% (v/v), and water, with a pH greater than 5 but less than 7. In a particular embodiment, the ketotifen formulation comprises ketotifen fumarate at concentration of 0.035% to 0.1% (w/v), or any specific value within said range. Preferably the ketotifen is ketotifen fumarate. In accordance with this embodiment, the ketotifen formulation does not contain a tear substitute, a polymeric, mucoadhesive vehicle, or hydrogen peroxide. Optionally, the formulation comprises a preservative other than hydrogen peroxide, preferably benzalkonium chloride at a concentration of from 0.005% to 0.02% (w/v) (or any specific value within said range), or stabilised oxychloro complex)(Purite°. In a particular embodiment, the ketotifen formulation comprises ketotifen at concentration of 0.1% (w/v), glycerol at 2% to 3% (v/v), and water. In another particular embodiment, the ketotifen formulation comprises ketotifen at a concentration of 0.035% (w/v), glycerol at 1% to 2% (v/v) and water. In certain embodiments, the formulation further comprises benzalkonium chloride at 0.01% (w/v). In certain embodiments, the pH of the formulation is between 5.5 and 6.5. In particular embodiments, the pH of the formulation is 5.2, 5.5, 6, or 6.5.

In a particular embodiment, the ketotifen formulation comprises 0.481 mg/mL ketotifen fumarate (equivalent to 0.350 mg/mL ketotifen base) as the only active agent in the formulation, 0.10 mg/mL benzalkonium chloride, 21.25 mg/mL glycerol, pH adjusted to 5.5, and purified water q.s. to 1 mL, wherein the formulation does not comprise a tear substitute, a polymeric, mucoadhesive vehicle, or hydrogen peroxide, and wherein the osmolality of the formulation is 255 mOsm/kg. In another particular embodiment, the ketotifen formulation comprises 0.481 mg/mL ketotifen fumarate (equivalent to 0.350 mg/mL ketotifen base) as the only active agent in the formulation for treating and preventing ocular allergy, particularly allergic conjunctivitis, 0.10 mg/mL benzalkonium chloride, 28.75 mg/mL glycerol, pH adjusted to 5.5, and purified water q.s. to 1 mL, wherein the formulation does not comprise a tear substitute, a polymeric, mucoadhesive vehicle, or hydrogen peroxide, and wherein the osmolality of the formulation is 345 mOsm/kg.

In one embodiment, the ketotifen formulation comprises ketotifen, or a pharmaceutically acceptable salt thereof, as the only active ingredient at a concentration of from 0.01% to 0.20% (w/v) (or any specific value within said range) and one or more tear substitutes or components thereof. In a particular embodiment, the ketotifen formulation comprises ketotifen fumarate at concentration of 0.035% to 0.1% (w/v), or any specific value within said range, and one or more tear substitutes or components thereof. Preferably the ketotifen is ketotifen fumarate. Preferably, the tear substitute or component thereof contains hydroxypropylmethyl cellulose or carboxymethyl cellulose or both. In some embodiments, the hydroxypropylmethyl cellulose or carboxymethyl cellulose is present at a concentration of 0.5% to 1% (w/v) (or any specific value within said range) and the resulting viscosity of the solution is 60-80 cpi. In a particular embodiment, the hydroxypropylmethyl cellulose or carboxymethyl cellulose is present at a concentration of 0.7% to 0.9%. Optionally, the formulation also comprises a preservative, preferably benzalkonium chloride at a concentration of from 0.005% to 0.02% (w/v) (or any specific value within said range) or stabilised oxychloro complex (Purite®). The pH of the formulation is preferably between 4 and 7. In certain embodiments, the pH of the formulation is between 5.5 and 6.5. In particular embodiments, the pH of the formulation is 5.2, 5.5, 6, or 6.5. In one embodiment, the osmolality of the formulation is between 225 and 400 mOsm. In another embodiment, the osmolality of the formulation is between 400 and 875 mOsm.

In one embodiment, the ketotifen formulation comprises ketotifen (0.001% to 0.20% or any specific value within said range), or a pharmaceutically acceptable salt thereof, in combination with oxymetazoline. In particular embodiments, ketotifen is formulated at a concentration of 0.001%, 0.005%, 0.010%, 0.02%, 0.025%, 0.03%, 0.035%, 0.04%, 0.045%, 0.05%, 0.06%, 0.07%, 0.08%, 0.09%, 0.10%, 0.15%, or 0.20% in combination with oxymetazoline. In certain embodiments, the oxymetazoline is present in the formulation at a concentration of from between 0.001% and 0.2% (w/v), or any specific value within said range. The pH of the formulation of ketotifen and oxymetazoline is preferably between 4 and 7, most preferably between 5.5 and 6.5 (or any specific value within said ranges). In one embodiment, the osmolality of the formulation is between 225 and 400 mOsm (or any specific value within said range). In another embodiment, the osmolality of the formulation is between 400 and 875 mOsm (or any specific value within said range).

In one embodiment, the formulation of ketotifen and oxymetazoline formulation further comprises one or more tear substitutes. Preferably, the tear substitutes contains hydroxypropylmethyl cellulose or carboxymethyl cellulose or both. In some embodiments, the hydroxypropylmethyl cellulose or carboxymethyl cellulose is present at a concentration of 0.5% to 1% (w/v) (or any specific value within said range) and the resulting viscosity of the solution is 60-80 cpi (or any specific value within said range). In a particular embodiment, the hydroxypropylmethyl cellulose or carboxymethyl cellulose is present at a concentration of 0.7% to 0.9% (or any specific value within said range). Optionally, the formulation also comprises a preservative, preferably benzalkonium chloride at a concentration of from 0.005% to 0.02% (w/v) (or any specific value within said range) or stabilised oxychloro complex (Purite®). The pH of the formulation is preferably between 4 and 7, or any specific value within said range. In certain embodiments, the pH of the formulation is between 5.5 and 6.5, or any specific value within said range. In particular embodiments, the pH of the formulation is 5.2, 5.5, 6, or 6.5. In one embodiment, the osmolality of the formulation is between 225 and 400 mOsm, or any specific value within said range. In another embodiment, the osmolality of the formulation is between 400 and 875 mOsm, or any specific value within said range.

In one embodiment, the ketotifen formulation comprises ketotifen (0.03% to 0.20%, or any specific value within said range) in combination with napahzoline. In particular embodiments, ketotifen is formulated at a concentration of from 0.04% to 0.08%, from 0.08% to 0.10%, from 0.10% to 0.15%, or from 0.15% to 0.20% (or any specific value within said ranges) in combination with naphazoline. In other particular embodiments, ketotifen is formulated at a concentration of 0.001%, 0.005%, 0.010%, 0.02%, 0.025%, 0.03%, 0.035%, 0.04%, 0.045%, 0.05%, 0.06%, 0.07%, 0.08%, 0.09%, 0.10%, 0.15%, or 0.20% in combination with naphazoline. In certain embodiments, the pH of the formulation of ketotifen and naphazoline is greater than 5 and the osmolality is less than 400 mOsm. In other embodiments, the pH of the formulation is between 4.8 and 6.8, preferably between 5.5 and 6.5 (or any specific value within said range). Optionally, the formulation also comprises a preservative, preferably benzalkonium chloride at a concentration of from 0.005% to 0.02% (w/v) (or any specific value within said range), or stabilised oxychloro complex)(Purite®).

In one embodiment, the formulation of ketotifen and naphazoline formulation further comprises one or more tear substitutes, or components thereof. Preferably, the tear substitute or component thereof contains hydroxypropylmethyl cellulose or carboxymethyl cellulose or both. In some embodiments, the hydroxypropylmethyl cellulose or carboxymethyl cellulose is present at a concentration of 0.5% to 1% (w/v) (or any specific value within said range) and the resulting viscosity of the solution is 60-80 cpi, or any specific value within said range. In a particular embodiment, the hydroxypropylmethyl cellulose or carboxymethyl cellulose is present at a concentration of 0.7% to 0.9%, or any specific value within said range. Optionally, the formulation also comprises a preservative, preferably benzalkonium chloride at a concentration of from 0.005% to 0.02% (w/v) (or any specific value within said range) or stabilised oxychloro complex (Purite®). The pH of the formulation is greater than 5 and the osmolality of the formulation is less than 400 mOsm. Preferably, the pH of the formulation is between 5 and 7, most preferably between 5.5 and 6.5, or any specific value within said range. The osmolality is preferably between 225 to 390 mOsm, or any specific value within said range.

In one embodiment, the ketotifen formulation comprises ketotifen (0.001% to 0.20%, or any specific value within said range) in combination with fluticasone. In particular embodiments, ketotifen is formulated at a concentration of 0.001%, 0.005%, 0.010%, 0.02%, 0.025%, 0.03%, 0.035%, 0.04%, 0.045%, 0.05%, 0.06%, 0.07%, 0.08%, 0.09%, 0.10%, 0.15%, or 0.20% in combination with fluticasone. In certain embodiments, the fluticasone is present in the formulation at a concentration of from between 0.001% and 0.2% (w/v), or any specific value within said range. Preferably, the fluticasone is present in the formulation at a concentration from between 0.001% and 0.01% (w/v), or any specific value within said range. In a particular embodiment, the fluticasone is present in the formulation at a concentration of 0.005% (w/v). The pH of the formulation of ketotifen and fluticasone is preferably between 4 and 7, most preferably between 5.5 and 6.5 (or any specific value within said range). In one embodiment, the osmolality of the formulation is between 225 and 400 mOsm (or any specific value within said range). In another embodiment, the osmolality of the formulation is between 400 and 875 mOsm (or any specific value within said range).

In one embodiment, the formulation of ketotifen and fluticasone formulation further comprises one or more tear substitutes, or components thereof. Preferably, the tear substitute or component thereof contains hydroxypropylmethyl cellulose or carboxymethyl cellulose or both. In some embodiments, the hydroxypropylmethyl cellulose or carboxymethyl cellulose is present at a concentration of 0.5% to 1% (w/v) (or any specific value within said range) and the resulting viscosity of the solution is 60-80 cpi, or any specific value within said range. In a particular embodiment, the hydroxypropylmethyl cellulose or carboxymethyl cellulose is present at a concentration of 0.7% to 0.9%, or any specific value within said range. Optionally, the formulation also comprises a preservative, preferably benzalkonium chloride at a concentration of from 0.005% to 0.02% (w/v) (or any specific value within said range) or stabilised oxychloro complex)(Purite®). The pH of the formulation is preferably between 4 and 7, or any specific value within said range. In one embodiment, the osmolality of the formulation is between 225 and 400 mOsm, or any specific value within said range. In another embodiment, the osmolality of the formulation is between 400 and 875 mOsm, or any specific value within said range.

### Stability

The formulations of the present invention provide for the chemical stability of the formulated ketotifen and other optional active agents of the formulation. “Stability” and “stable” in this context refers to the resistance of the ketotifen and other optional active agents to chemical degradation under given manufacturing, preparation, transportation and storage conditions. The “stable” formulations of the invention also preferably retain at least 90%, 95%, 98%, 99%, or 99.5% of a starting or reference amount under given manufacturing, preparation, transportation, and/or storage conditions. The amount of ketotifen and other optional active agents can be determined using any art-recognized method, for example, as UV-Vis spectrophotometry and high pressure liquid chromatography (HPLC).

In certain embodiments, the ketotifen formulations are stable at temperatures ranging from about 20 to 30° C. for at least 1 week, at least 2 weeks, at least 3 weeks, at least 4 weeks, at least 5 weeks, at least 6 weeks, or at least 7 weeks. In other embodiments, the formulations are stable at temperatures ranging from about 20 to 30° C. for at least 1 month, at least 2 months, at least 3 months, at least 4 months, at least 5 months, at least 6 months, at least 7 months, at least 8 months, at least 9 months, at least 10 months, at least 11 months, or at least 12 months. In one embodiment, the formulation is stable for at least 3 months at 20-25° C.

In other embodiments, the ketotifen formulations are stable at temperatures ranging from about 2 to 8° C. for at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the formulation is stable for at least 2 months at 2 to 8° C.

In other embodiments, the ketotifen formulations are stable at temperatures of about −20° C. for at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the formulation is stable for at least 6-12 months at −20° C.

In a particular embodiment, a ketotifen formulation of the invention is stable at temperatures of about 20-30° C. at concentrations up to 0.10% for at least 3 months. In another embodiment, the formulation is stable at temperatures from about 2-8° C. at concentrations up to 0.10% for at least 6 months.

### Methods of Use

The ketotifen formulations of the invention are useful for the treatment and prevention of the symptoms of ocular allergy, such as ocular itching, redness, and eyelid swelling, as well as associated nasal symptoms. In a preferred embodiment, the present invention provides a 0.035% ketotifen formulation which is comfortable and effective to relieve ocular itching when administered once a day to the eye.

The invention provides methods of treating and preventing ocular allergy in a subject in need thereof comprising administering to the eye surface of the subject a pharmaceutical composition comprising an effective amount of ketotifen. In certain embodiments, the administration of ketotifen to the eye of a subject in need of treatment and prevention of ocular allergy is also effective to mitigate or reduce one or more nasal symptoms associated with the allergy. The subject is preferably a human, but may be another mammal, for example a dog, a cat, a rabbit, a mouse, a rat, or a non-human primate.

The term “effective amount” means an amount of ketotifen that is sufficient to eliminate or reduce a symptom of ocular allergy. In certain embodiments, the effective amount is the amount sufficient for the treatment and prevention of ocular allergy. “Treatment” in this context refers to reducing or ameliorating at least one symptom of ocular allergy. “Prevention” in this context refers to a reduction in the frequency of, or a delay in the onset of, symptoms associated with ocular allergy, relative to a subject who does not receive the composition. The effective amount of ketotifen and other active agents in the formulation will depend on absorption, inactivation, and excretion rates of the drug as well as the delivery rate of the compound from the formulation. Particular dosages may also vary with the severity of the condition to be alleviated. It is to be further understood that for any particular subject, specific dosage regimens should be adjusted over time according to the individual need and the professional judgment of the person administering or supervising the administration of the compositions. Typically, a dosing regiment will be determined using techniques known to one skilled in the art.

Examples of dosing regimens that can be used in the methods of the invention include, but are not limited to, once daily, twice daily, three times, and four times daily. In a preferred embodiment, the method comprises administering a ketotifen formulation of the invention to the eye of the subject once a day. In some embodiments, the administration is 2 to 4 times a day.

In certain embodiments, once a day administration (q.d.) is effective to mitigate the symptoms of ocular allergy. However, particular dosages may also selected based on a number of factors including the age, sex, species and condition of the subject. Effective amounts can also be extrapolated from dose-response curves derived from in vitro test systems or from animal models.

In a preferred embodiment, the formulations of the present invention contain an effective amount of ketotifen (or a pharmaceutically acceptable salt thereof) as the only active agent for treating and preventing ocular allergy. In particular embodiment, the method for treating and preventing ocular allergy in a subject in need thereof comprises topically administering to the eye of a subject an ophthalmic formulation comprising ketotifen (or a pharmaceutically acceptable salt thereof) as the only active agent in the formulation for treating and preventing ocular allergy, glycerol, and benzalkonium chloride, wherein the concentration of ketotifen is from 0.01% to 0.20%, preferably from 0.02% to 0.04% (w/v) (or any specific value within said range), the concentration of glycerol in the formulation is from 2% to 3% (v/v), or any specific value within said range, the pH of the formulation is between 5.0 and 7, preferably between 5.0 and 6.5, and most preferably about 5.5, the osmolality of the formulation is greater than 290 mOsm and less than 400 mOsm, and the formulation does not comprise a tear substitute, a polymeric, mucoadhesive vehicle, or hydrogen peroxide.

In another particular embodiment, the method for treating and preventing ocular allergy comprises administering to the eye of a subject in need thereof an ophthalmic formulation comprising an effective amount of ketotifen, or a pharmaceutically acceptable salt thereof, wherein the concentration of ketotifen is about 0.035% (w/v), the pH of the formulation is about 5.5, the osmolality of the formulation is between 300-350 mOsm (or any specific value within said range), and the formulation does not comprise a tear substitute, hydrogen peroxide, or any active ingredient other than ketotifen.

For example, the method for treating and preventing ocular allergy in a subject in need thereof comprises topically administering to the eye of a subject a ketotifen formulation comprising 0.481 mg/mL ketotifen fumarate (equivalent to 0.350 mg/mL ketotifen base) as the only active agent in the formulation for treating and preventing ocular allergy, 0.10 mg/mL benzalkonium chloride, 21.25 mg/mL glycerol, pH adjusted to 5.5, and purified water q.s. to 1 mL, wherein the formulation does not comprise a tear substitute, a polymeric, mucoadhesive vehicle, or hydrogen peroxide, and wherein the osmolality of the formulation is 255 mOsm/kg. In further example, the method for treating and preventing ocular allergy in a subject in need thereof comprises topically administering to the eye of a subject a ketotifen formulation comprising 0.481 mg/mL ketotifen fumarate (equivalent to 0.350 mg/mL ketotifen base) as the only active agent in the formulation for treating and preventing ocular allergy, 0.10 mg/mL benzalkonium chloride, 28.75 mg/mL glycerol, pH adjusted to 5.5, and purified water q.s. to 1 mL, wherein the formulation does not comprise a tear substitute, a polymeric, mucoadhesive vehicle, or hydrogen peroxide, and wherein the osmolality of the formulation is 345 mOsm/kg.

Optionally, one or more additional active ingredients, that are effective for the intended use (i.e. to mitigate the symptoms of ocular allergy) may be combined with ketotifen for the treatment and prevention of ocular allergy. The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

In one embodiment, ketotifen is formulated with one or more of oxymetazoline, naphazoline, and fluticasone and administered to the eye of a subject in need of treatment and prevention of an ocular allergy once daily. In certain embodiments, the combination formulation is administered one, two or three times a day.

### Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

### Kits

The present invention provides a pharmaceutical pack or kit comprising one or more containers filled with a liquid or lyophilized ketotifen formulation of the invention. In one embodiment, the formulation is an aqueous formulation of ketotifen. In one embodiment, the formulation is lyophilized. In preferred embodiments the liquid or lyophilized formulation is sterile. In one embodiment, the kit comprises a liquid or lyophilized formulation of the invention, in one or more containers, and one or more other prophylactic or therapeutic agents useful for the treatment and prevention of ocular allergy. The one or more other prophylactic or therapeutic agents may be in the same container as the ketotifen or in one or more other containers. Preferably, the ketotifen is formulated at a concentration of from about 0.25% (w/v) to about 1.0% (w/v) (or any specific value within said ranges) and is suitable for topical ocular administration. In certain embodiments, the kit contains the ketotifen in unit dosage form.

In certain embodiments, the kit further comprises instructions for use in the treatment and prevention of ocular allergy (e.g., using the ketotifen formulations of the invention alone or in combination with another prophylactic or therapeutic agent), as well as side effects and dosage information for one or more routes of administration. Optionally associated with such container(s) is a notice in the form prescribed by a governmental agency regulating the manufacture, use or sale of pharmaceuticals or biological products, which notice reflects approval by the agency of manufacture, use or sale for human administration. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

In another embodiment, this invention provides kits for the packaging and/or storage and/or use of the formulations described herein, as well as kits for the practice of the methods described herein. The kits can be designed to facilitate one or more aspects of shipping, use, and storage.

All publications and patents mentioned herein are hereby incorporated by reference in their entirety as if each individual publication or patent was specifically and individually indicated to be incorporated by reference.

### EXAMPLES

The invention is further defined by reference to the following examples, which are not meant to limit the scope of the present invention. It will be apparent to those skilled in the art that many modifications, both to the materials and methods, may be practiced without departing from the purpose and interest of the invention.

### Example 1

A phase 2 double masked, placebo controlled, clinical trial was conducted to evaluate the efficacy of a ketotifen 0.035% ophthalmic formulation (N=16) (shown in Table 1) compared to vehicle (N=16). The unit quantity of ketotifen fumarate, benzalkonium chloride and glycerol, shown in Table 1, are each indicated in mg/ml.

TABLE 1
ketotifen 0.035% ophthalmic formulation

| Quantity (mg/mL) | Raw Material |
|---|---|
| 0.481 | Ketotifen Fumarate Ph. Eur.* [Sifavitor S.P.A] |
| (0.350) | (Equivalent to Ketotifen base) |
| 0.10 | Benzalkonium chloride, NF |
| 21.25 | Glycerol, USP |
| Adjust pH to 5.5 | Sodium hydroxide, 0.5N or Hydrochloric Acid, 0.5N |
| q.s. to 1 mL | Purified Water, USP |

The osmolality of the formulation shown in Table 1 was determined by freezing point depression following USP <785>. The osmolality of the formulation shown in Table 1 was determined to be 255 mOsm/kg.

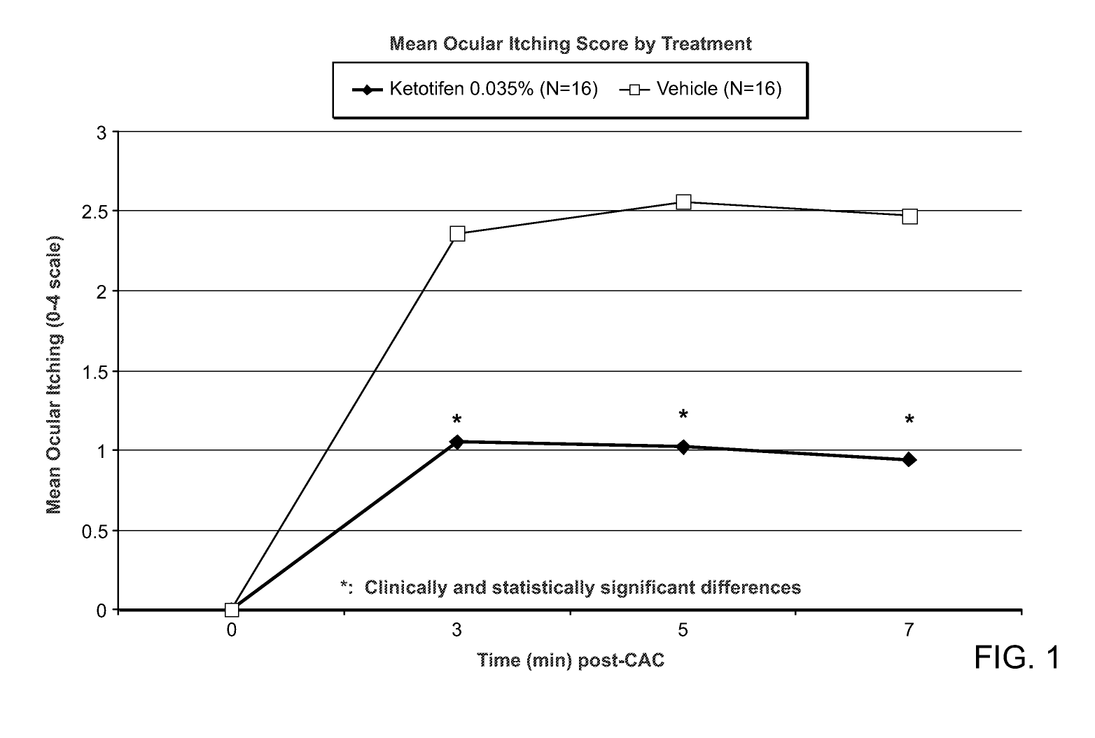

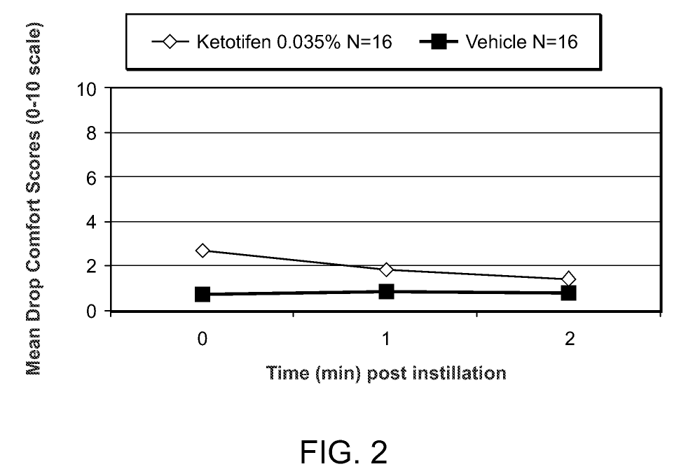

Subjects underwent 2 screening visits (an allergen titration and confirmation) followed by a drug evaluation visit. At the drug evaluation visit, one drop of masked study medication was instilled in both eyes and comfort assessments were taken. Sixteen hours later subjects were challenged with allergen and allergic assessments were taken. Subjects were asked to subjectively rate their ocular itching on a scale of 0 to 4 (0=little to no itching, 4=extreme itching) The results demonstrate that one drop of ketotifen 0.035% ophthalmic solution prevented ocular itching associated with ocular allergy when administered 16 hours prior to conjunctival allergen challenge (CAC) (see Table 2 and FIG. 1). Differences between the group receiving the ketotifen solution and that receiving vehicle were clinically (≧1 unit difference) and statistically significant (P<0.05). Comfort was also subjectively assessed by each subject following administration of the drop on a scale 0-10 (0=extremely comfortable, 10=extremely uncomfortable). As shown in FIG. 2, the 0.035% ketotifen solution was very comfortable and well tolerated upon instillation in the eye.

The results were surprising in that the formulation was stable, comfortable, and had efficacy supporting a duration of action of 16 hours, which is indicative of once daily dosing. This efficacy is superior to that of currently marketed 0.025% ketotifen (approved for twice daily dosing) based on historical data (Berdy et al Clinical Ther 2002; Zaditor FDA Summary Basis of Approval) since only one dose per day is required and yet the 0.035% solution was just as comfortable as the marketed 0.025% solution. It is also superior to the 0.05% solution currently marketed in Japan which has a low comfort level and is only approved for BID (i.e., twice daily) and QID (i.e., 4 times a day) dosing. There was also a statistically significant reduction in nasal congestion with ketotifen 0.035% compared with placebo at 7 min post-CAC.

TABLE 2
Mean Ocular Itching Scores (0-4 scale)
following CAC 16 hrs after dosing

| | | | | Mean | |
|---|---|---|---|---|---|
| | | Ketotifen | | Difference | |
| | | 0.035% HCl | Vehicle | (ketotifen- | |
| Statistic | Timepoint | (N = 16) | (N = 16) | vehicle) | p-value |
| Mean | Pre-CAC | 0.00 (0.00) | 0.00 (0.00) | 0.00 | 1.0000 |
| (SD) | 3 min | 1.05 (0.87) | 2.36 (0.58) | −1.31 | <0.0001 |
| | 5 min | 1.02 (0.62) | 2.56 (0.60) | −1.54 | <0.0001 |
| | 7 min | 0.94 (0.61) | 2.47 (0.72) | −1.53 | <0.0001 |

### Example 2

A double masked, placebo controlled, clinical trial was conducted to evaluate the efficacy of another ketotifen 0.035% ophthalmic formulation (N=4) (shown in Table 3) compared to vehicle (N=4). The unit quantity of ketotifen fumarate, benzalkonium chloride and glycerol, shown in Table 3, are each indicated in mg/ml.

TABLE 3
ketotifen 0.035% ophthalmic formulation

| Quantity (mg/mL) | Raw Material |
|---|---|
| 0.481 | Ketotifen Fumarate, Ph. Eur.* [Sifavitor S.P.A] |
| (0.350) | (Equivalent to Ketotifen base) |
| 0.10 | Benzalkonium chloride, NF |
| 28.75 | Glycerol, USP |
| Adjust pH to 5.5 | Sodium hydroxide, 0.5N or Hydrochloric Acid, 0.5N |
| q.s. to 1 mL | Purified Water, USP |

The osmolality of the formulation shown in Table 3 was determined by freezing point depression following USP <785>. The osmolality of the formulation shown in Table 3 was determined to be 345 mOsm/kg.

Subjects who were known to have an allergic response with a known allergen dose, underwent a drug evaluation visit. At the drug evaluation visit, one drop of masked study medication was instilled in both eyes and comfort assessments were taken. Sixteen hours later subjects were challenged with allergen and allergic assessments of ocular itching and conjunctival redness, were taken.

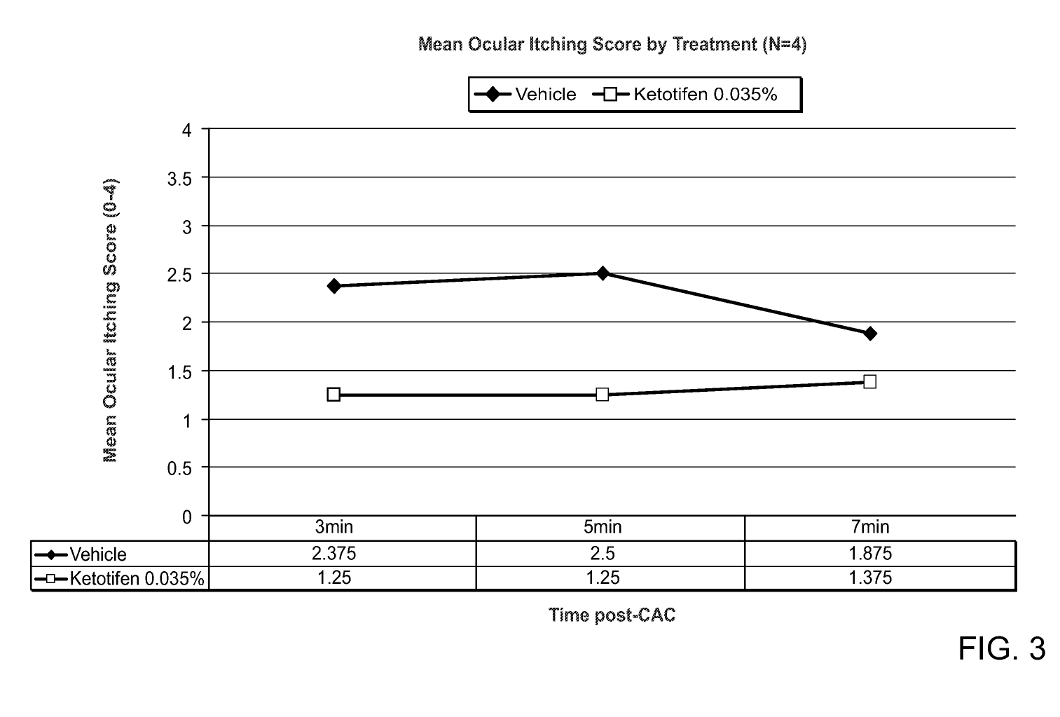

The results demonstrate that one drop of ketotifen 0.035% ophthalmic solution prevented ocular itching associated with ocular allergy when administered 16 hours prior to conjunctival allergen challenge (CAC) (see FIG. 3). Differences between the group receiving the ketotifen solution and that receiving vehicle were clinically (≧1 unit difference) and statistically significant (P<0.05).

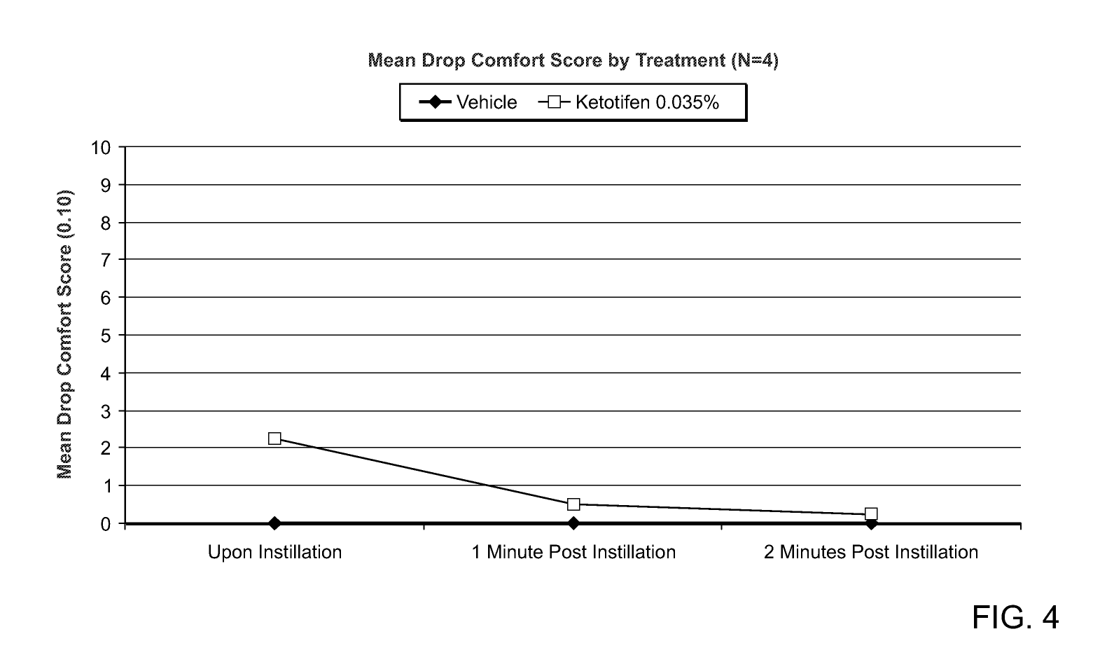

Tolerability and drop comfort were also assessed following administration of the drop on a scale 0-10 (0=extremely comfortable and 10=extremely uncomfortable). As shown in FIG. 4, the 0.035% ketotifen solution was very comfortable and well tolerated upon instillation in the eye.

The results were surprising in that the formulation was stable, extremely comfortable, and had efficacy supporting a duration of action of at least 16 hours and up to 24 hours, indicative of only once daily dosing. In comparison, the currently marketed 0.025% ketotifen solution, commercially known as Zaditor™ (0.025% ketotifen, pH 5.5, 210-300 mOsm/kg), which is only approved for twice daily dosing, only shows efficacy in reducing ocular itching for up to 12 hours in a comparable CAC model (see Center for Drug Evaluation and Research, NDA 21-066, Ketotifen fumarate ophthalmic solution, page 22 (Dec. 31, 1998)). In further comparison the 0.05% ketotifen formulation currently marketed in Japan has a low comfort/tolerability level and is only approved for BID (i.e., twice daily) and QID (i.e., four times a day) dosing. These results unexepectedly demonstrate that a marginal increase in concentration of ketotifen (i.e., 0.035% ketotifen as used herein vs. Zaditor™ (0.025% ketotifen)) produces more than a marginal increase in efficacy, as defined by hours of itching relief (at least 16 and up to 24 hours). It is additionally surprising that the preferred 0.035% ketotifen concentration described and tested herein compares most favorably to currently sold ketotifen products with as much as 2× the API concentration (0.05%), and the prior 0.05% formulations were not shown to not provide benefit over 0.025%, whereas the formulation described herein supports QD dosing (i.e., once a day dosing).

Furthermore, historical data shows that a dose-response relationship in the efficacy of reducing itching was not observed for ketotifen ophthalmic solutions ranging in concentration from 0.025%, 0.05%, 0.1%, and 0.15%. This same efficacy analysis of the dose-ranging study supported the choice of 0.025% ketotifen for Zaditor™, as it was shown to be the lowest concentration that was efficacious (see Center for Drug Evaluation and Research, NDA 21-066, Ketotifen fumarate ophthalmic solution, page 14 (Dec. 31, 1998)). As such, the superior 16 hour efficacy achieved using one drop of the ketotifen 0.035% ophthalmic solution described herein was a surprising and unexpected result in view of the historical data generated for the approval of Zaditor™.

Moreover, the 16 hour efficacy using the 0.035% ketotifen solution described herein, is superior to the efficacy achieved using the 0.025% ketotifen solution, Zaditor, at 6 and 8 hour timepoints (see Center for Drug Evaluation and Research, NDA 21-066, Ketotifen fumarate ophthalmic solution, page 22 (Dec. 31, 1998)).

It is furthermore surprising and unexpected that a 0.035% ketotifen solution having a low pH (i.e., pH 5.5) would be so extremely comfortable upon instillation in the eye, without the inclusion of a tear substitute component, or other masking agent, to prevent the stinging/burning sensation which is commonly known in the art to be associated with higher concentrations of ketotifen. Without intending to be bound by theory, the superior properties of the 0.035% formulations described herein are attributed, in part, to the identification of an optimal ketotifen concentration (i.e., 0.035%) and optimal osmolality (345 mOsm/kg), which in combination provide superior comfort and efficacy. Comfort provided by the optimal osmolality lends to the increased efficacy over prior commercial formulations by increasing the dwell time of the ketotifen in the eye.

In summary, the 0.035% ketotifen formulations described herein have unexpectedly superior efficacy and comfort profiles to that of currently marketed 0.025% ketotifen (approved for twice daily dosing) based on historical data (Berdy et al Clinical Ther 2002; Zaditor FDA Summary Basis of Approval) since only one dose per day is required and yet the 0.035% solution was just as comfortable as the marketed 0.025% solution. The comfort and efficacy of the 0.035% ketotifen formulations described herein are also superior to the 0.05% solution currently marketed in Japan which has a low comfort level and is only approved for BID (i.e., twice daily) and QID (i.e., four times a day) dosing.

### EQUIVALENTS

Those skilled in the art will recognize, or be able to ascertain using no more than routine experimentation, many equivalents to the specific embodiments of the invention described herein. Such equivalents are intended to be encompassed by the following claims. claims:

What is claimed is:

1. An ophthalmic formulation comprising ketotifen as the only active agent in the formulation, or a pharmaceutically acceptable salt thereof, wherein the concentration of ketotifen is from 0.01% to 0.20% (w/v), the pH of the formulation is between 5 and 6.5, the formulation does not comprise a tear substitute or hydrogen peroxide, and wherein the osmolality of the formulation is greater than 290 mOsm and less than 400 mOsm.

2. The ophthalmic formulation of claim 1, wherein the concentration of ketotifen is from 0.02% to 0.04%.

3. The ophthalmic formulation of claim 2, wherein the concentration of ketotifen is 0.035%.

4. The ophthalmic formulation of claim 1, wherein the ketotifen is in the form of ketotifen fumarate.

5. The ophthalmic formulation of claim 1, further comprising glycerol.

6. The ophthalmic formulation of claim 5, wherein the concentration of glycerol is from 2% to 3% (v/v).

7. The ophthalmic formulation of claim 6, further comprising benzalkonium chloride.

8. The ophthalmic formulation of claim 7, wherein the concentration of benzalkonium chloride is from 0.005% to 0.02% (v/v).

9. The ophthalmic formulation of claim 6, further comprising stabilized oxychloro complex.

10. An ophthalmic formulation comprising ketotifen as the only active agent in the formulation, or a pharmaceutically acceptable salt thereof, 2%-3% glycerol, and 0.01% benzalkonium chloride, wherein the concentration of ketotifen is from 0.035% (w/v), the pH of the formulation is between 5.5, the formulation does not comprise a tear substitute or hydrogen peroxide, and wherein the osmolality of the formulation is greater than 290 mOsm and less than 400 mOsm.

11. The ophthalmic formulation of claim 1, further comprising an additional active agent selected from the group consisting of naphazoline, oxymetazoline and fluticasone.

12. The ophthalmic formulation of claim 1, further comprising a tear substitute.

13. The ophthalmic formulation of claim 12, wherein the tear substitute is hydroxypropyl methylcellulose or carboxymethyl cellulose.

14. The ophthalmic formulation of claim 13, further comprising a preservative, wherein the preservative is benzalkonium chloride or stabilized oxychloro complex)((Purite®).

15. The ophthalmic formulation of claim 1, further comprising a mucoadhesive, polymeric vehicle.

16. The ophthalmic formulation of claim 15, wherein the mucoadhesive, polymeric vehicle is Durasite®.

17. The ophthalmic formulation of claim 1, wherein the pH of the formulation is 5.5.

18. The ophthalmic formulation of claim 1, wherein the formulation is an aqueous formulation.

19. The ophthalmic formulation of claim 1, wherein the formulation is in the form of a single dose unit.

20. The ophthalmic formulation of claim 19, wherein the formulation does not comprise a preservative.

21. A method for treating and preventing ocular allergy by topically administering to the eye of a subject in need thereof an ophthalmic formulation comprising ketotifen as the only active agent in the formulation, or a pharmaceutically acceptable salt thereof, wherein the concentration of ketotifen is from 0.01% to 0.20% (w/v), the pH of the formulation is between 5 and 6.5, the formulation does not comprise a tear substitute or hydrogen peroxide, and wherein the osmolality of the formulation is greater than 290 mOsm and less than 400 mOsm.

22. The method of claim 21, wherein the ocular allergy is allergic conjunctivitis.

23. The method of claim 21, wherein the ophthalmic formulation is administered once daily.

24. The method of claim 21, wherein the ketotifen is in the form of ketotifen fumarate.

25. The method of claim 21, wherein the ophthalmic formulation further comprises an active agent selected from the group consisting of naphazoline, oxymetazoline, or fluticasone.
